(19) United States
(12) Patent Application Publication
(10) Pub. No.: US 2025/0380892 A1
(43) Pub. Date: Dec. 18, 2025

(54) METHOD TO OBTAIN A SKIN ELECTRODE PATCH AND RESPECTIVE SKIN ELECTRODE PATCH

(71) Applicant: UNIVERSIDADE DE COIMBRA, Coimbra (PT)

(72) Inventors: Manuel Francisco REIS CARNEIRO, Coimbra (PT); Mahmoud TAVAKOLI, Coimbra (PT); Carmel MAJIDI, PITTSBURGH, PA (US); Pedro Filipe ALHAIS LOPES, LAGOA (PT)

(73) Assignee: UNIVERSIDADE DE COIMBRA, Coimbra (PT)

(21) Appl. No.: 18/880,121

(22) PCT Filed: Jun. 30, 2023
(86) PCT No.: PCT/IB2023/056853

§ 371 Date: Dec. 30, 2024

(30) Foreign Application Priority Data
Jun. 30, 2022 (PT) 118076

(51) Int. Cl. A61B 5/263; A61B 5/00; A61B 5/257

(52) U.S. Cl. CPC A61B 5/263; A61B 5/257; A61B 5/68335; A61B 2562/125; A61B 2562/166

(57) ABSTRACT

The present disclosure relates to a method for obtaining a skin electrode patch and said skin electrode. One method according to the disclosure includes the steps of: applying conductive ink to a first polymeric layer; cutting one or more cutouts, on a second polymeric layer, for the conductive ink to contact with the skin-; arranging as consecutive layers: the first polymeric layer; the conductive ink; a flexible printed circuit board, PCB; the second polymeric layer; heat-pressing the arranged layers; wherein the conductive ink is configured to be exposed through said one or more cut-outs to the skin and to connect with the flexible PCB.

### TECHNICAL FIELD

The present disclosure relates to a method to obtain a skin electrode patch and the respective skin electrode patch.

### BACKGROUND

Health monitoring systems have undergone significant developments in the last decade, mainly thanks to the advent of wearable technology, which contrasts with the bulky, rigid, and mainly thanks to the advent of wearable technology, which contrasts with the bulky, rigid, and obstructive equipment commonly found in clinics and hospitals [1]. In the last few years wearable devices were proposed for long-term and continuous electrophysiological monitoring. This includes both e-textiles or ultrathin adhesive e-skins for the recording of biopotentials (electrocardiography—ECG [2-4],electromyography—EMG [5-7], electroencephalography—EEG [8-11], or electrooculography—EOG [7,10,12]), respiration rate [13-15], temperature [16], pulse oximetry [17,18], or human motion [19,20]. The ultimate goal is to enable long-term electrophysiological monitoring, increase the patient comfort during such analysis, and to reduce the healthcare burden by enabling remote patient monitoring-an aspect to which more importance has been given after the recent COVID-19 pandemic [21]. It also allows patients to be monitored 24/7 while remaining in their homes and keeping their routines, which has been proven to speed-up recoveries while reducing healthcare costs [22,23].

This has been a major driving force for the novel field of stretchable and thin-film electronics, that intends to develop advanced electronic systems that can adapt to the dynamic morphology of the human skin. Materials and fabrication techniques for advancement of this field have been a major focus of investigation in the past few years [24,25]. This includes techniques for implementation of deterministic structures [26], conductive composites [27-29], and liquid metals [30,31], which include soft lithography [32,33], laser patterning [34-36], stencil printing [10,33], or direct deposition [13,27,28,36]. Another requirement is the integration of skin-interfacing electrodes to collect biopotentials from the epidermis for EMG/ECG/EEG monitoring, which include the traditional Ag/AgCl electrodes, novel wet hydrogel electrodes [37,38] as well as printed dry electrodes based on carbon [10], silver [2,10,16] or polymers such as PEDOT:PSS [39]. Incorporation of thin-film printed electrodes generally is preferable due to higher user comfort, easier implementation, a more desirable form factor, and the possibility of very low-cost and scalable fabrication. However, compared to gel electrodes, these printed dry electrodes suffer from a lower skin-conformance, a higher electrode-skin impedance and thus a lower signal quality [40].

In summary, implementation of a truly wearable, comfortable, thin-film, and low-cost electrophysiological monitoring system that provides a medical-grade interfacing quality is still not demonstrated. A few implementations of “electronic tattoos” [4,12,17,27] are valuable progress, however these usually fall short either in terms of the required robustness for long-term monitoring, or in terms of signal quality that usually gets degraded over time due to reduced electrode-skin conformance. As well, despite the progresses, there have been limited efforts to create fully-functional systems—instead, most studies focus on the synthesis and characterization of standalone electrodes [8,11,12,37-39]. Furthermore, in cases where a fully standalone patch is presented, it is typically limited to a single predefined application [3,6,10,16].

These facts are disclosed in order to illustrate the technical problem addressed by the present disclosure.

### GENERAL DESCRIPTION

It is disclosed a novel architecture of materials and fabrication techniques that serves as a universal method for implementation of thin-film biostickers, preferably a skin electrode patch, for high resolution electrophysiological monitoring. Unlike the existing wearable patches, the presented disclosure can be worn for several days, and is not affected by daily routines such as physical exercise or taking bath. The present disclosure comprises a printable biphasic liquid metal silver composite, both as the electrical interconnects and the electrodes. This allows combining advantages of dry electrodes i.e. printability and non-smearing behaviour, with benefits of wet electrodes i.e. high quality signal. A study showed that these biphasic printed electrodes benefit from a lower electrode-skin impedance compared to clinical grade Ag/AgCl electrodes. Digital printing enables autonomous fabrication of biostickers that are taylor-made for each user and each application. It was also developed a universal miniaturized electronic system for biopotential acquisition and wireless communication, and demonstrated multiple biopotential acquisition cases, including ECG, EEG, EMG, and EOG.

The present disclosure relates to a method for obtaining a skin electrode patch comprising the steps of:
applying conductive ink to a first polymeric layer;
cutting one or more cut-outs, on a second polymeric layer, for the conductive ink to contact with the skin;
arranging as consecutive layers:
   the first polymeric layer;
   the conductive ink;
   a flexible printed circuit board, PCB;
   the second polymeric layer;
heat-pressing the arranged layers;
wherein the conductive ink is configured to be exposed through said one or more cut-outs to the skin and to connect with the flexible PCB.

In an embodiment, the method further comprises arranging an adhesive layer on said second polymeric layer, said adhesive layer having one or more cut-outs for the conductive ink to contact with the skin.

In an embodiment, the method further comprises arranging said adhesive layer as a consecutive layer on the second polymeric layer before heat-pressing the arranged layers.

In an embodiment, the heat-pressing is performed with a textile heat press.

In an embodiment, the temperature of the heat-pressing is at least 150° C., in particular 150° C.

In an embodiment, the method further comprises arranging a rigid printed circuit board, PCB, on the flexible printed circuit board, in particular between the flexible printed circuit board and the first polymeric layer.

In an embodiment, the cutting is performed by a laser, preferably a CO₂ laser.

In an embodiment, the first polymeric layer and the second polymeric layer are made of thermoplastic polyurethane.

In an embodiment, the conductive ink is a Silver—Indium—Gallium—Styrene-Isoprene Copolymer, Ag—In—Ga-SIS, ink.

It is also disclosed a skin electrode patch comprising consecutive heat-pressed layers of:
a first polymeric layer;
conductive ink;
a flexible printed circuit board, PCB;
a second polymeric layer;
wherein the second polymeric layer has one or more cut-outs for the conductive ink to contact with the skin;
wherein the conductive ink is configured to be exposed through said one or more cut-outs to the skin and to connect with the flexible PCB.

In an embodiment, the patch further comprises an adhesive layer on said second polymeric layer, having one or more cut-outs for the conductive ink to contact with the skin.

In an embodiment, the cut-outs of the first polymeric layer correspond to the cut-outs of the adhesive layer, in particular between the flexible printed circuit board and the first polymeric layer.

In an embodiment, a rigid printed circuit board, PCB, is arranged on the flexible printed circuit board.

In an embodiment, the first polymeric layer and the second polymeric layer has a thickness of 50 μm each.

In an embodiment, the adhesive layer has a thickness of 60 μm.

In an embodiment, the conductive ink is a Silver—Indium—Gallium—Styrene-Isoprene Copolymer, Ag—In—Ga-SIS ink.

In an embodiment, the first polymer layer and the second polymer layer are made of thermoplastic polyurethane.

### BRIEF DESCRIPTION OF THE DRAWINGS

The following figures provide preferred embodiments for illustrating the disclosure and should not be seen as limiting the scope of invention.

FIG. 1: Schematic representation of an embodiment of a patch wherein. Depending on its shape and placement in the body, the proposed adhesive patches can be used for detection of multiple electrophysiological signals: brain waves (EEG), eye movement (EOG), neuromuscular activity (EMG), cardiac activity (ECG), and respiration. A: The various layers and components that compose the e-patch. B: Rigid analog front end printed circuit board.

FIG. 2: Schematic representation of an embodiment of a patch wherein skin wrinkles are imprinted on the adhesive interface layer of the patch.

FIG. 3: Illustration of results of a patch wherein A: Ag—In—Ga-SIS patch (top) and Ag/AgCl electrodes (bottom) placed on user's ventral forearm. B: Bode plots of the electrode-skin impedance for different electrode types, averaged across the measurements for subject 1 to 9. C: Average impedance in the EEG range (1-100 Hz), averaged for subjects 1 to 9. D: Electrode-skin interface equivalent circuit. E: Equivalent circuit's parameters average for subjects 1 to 9 and maximum/minimum error bars.

FIG. 4: Illustration of results of aging an embodiment of patch wherein A: Variation of ink resistance over 3 days for 5 samples, while performing everyday activities such as sleeping, jogging, and showering. B: Patch with printed Ag—In—Ga-SIS conductive lines with flexible contacts for easy measurement of resistance, adhered to the subject's abdomen (t=0 h). C: Wet patch after showering (t=24 h). D: Patch right before removal (t=73 h). E: skin redness after patch removal. F: Evolution of electrode-skin impedance over the course of 3 days for standard Ag/AgCl electrodes and printed Ag—In—Ga-SIS electrodes (averaged for 3 measurements). G: Average electrode-skin impedance for Ag/AgCl electrodes and printed Ag—In—Ga-SIS electrodes in the EEG range over the course of 3 days (averaged for 3 measurements).

FIG. 5: Illustration of results of an embodiment of the patch wherein A: Close-up image of the patch showing the two measuring electrodes for 1 Lead Electrocardiogramalog-front-end, and battery. B: Acquired ECG signal and identification of normal ECG features such as the QRS Complex and P and T waves. An R-R interval of 790 ms, from which a heartrate measurement of 76 bpm can be derived. C: Comparison between ECG signal on first day and after three days of the patch being used. Although Signal to Noise Ratio decreases, normal ECG features are still visible. D: Respiration signal can be derived from the ECG measurement thanks to the change in amplitude of the QRS complex during respiration cycles.

FIG. 6: Illustration of results of an embodiment of the patch wherein A: Position of ECG recording electrodes according to the EASI montage. B, C: Multielectrode ECG patch based on the EASI system. D: Position of the ECG recording electrodes in a conventional 12 lead system. E: Electrocardiogram signals directly acquired from the EASI patch (AS, ES, Al) and full 12 lead ECG derived from the EASI recordings (V1-V6, I, II, III, aVR, aVL, aVF).

FIG. 7: Illustration of results of a patch wherein A: EEG/EOG/EMG patch and identification of the electrodes. B: Placement of the patch in the user's face and forehead. C: Berger effect in EEG waves. When a user opens their eyes, the alpha rhythm is attenuated in amplitude. The higher amplitude of the acquired EEG signal when the user keeps their eyes closed can be observed in the plots. D: Eye movement detection through the EOG electrodes placed near the eyes. We can observe that the left and right electrodes show opposite polarities when lateral eye movement is detected. Eyeblinks, which are a predominantly vertical movement, are detected with higher amplitude by the EOG electrode placed below the user's eye. E: Muscle artifacts from masticatory movement detected recorded by the EMG electrode placed on top of the masseter muscle.

FIG. 8: Illustration of results of a patch wherein A: 16 electrode EMG patch. B: Setup for gripping force measurement with the EMG patch placed on the proximal portion of the anterior right forearm of the user, while grabbing a hand grip dynamometer. C: EMG signal (channel 4) after full-wave rectification for different gripping forces and averaged peak envelope. D: EMG signals acquired from 3 channels for 6 distinct hand poses (P1-P6). E: Channel positions and polarity of the electrodes on the EMG patch placed on the right forearm. F: Scaled average amplitude of the 8 EMG signals for 12 distinct hand poses (P1-P12).

FIG. 9: Illustration of results that shows the electrode skin impedance measured for the 3 electrode types tested in each subject.

FIG. 10: Illustration of results that shows the average electrode-skin impedance (1-100 Hz) in the EEG range for each electrode material in each subject.

FIG. 11: Illustration of results that is the calculated components (Rs. Rd, Cd) from the electrode-skin interface equivalent circuit, for each electrode type tested in each subject.

FIG. 12: Illustration of results that depicts the 3 impedance measurements, each taken over the course of 3 days with 3 distinct electrode sets for the same user.

FIG. 13: Illustration of results that shows the variability analysis among the 12 poses wherein A: Variability analysis among the 12 poses. The plot shows the dissimilarity coefficient between pairs of gestures. B: Similarity analysis for each pose. The plot shows the average correlation coefficient for each gesture across three repetitions.

FIG. 14: Schematic representation of the fabrication process of the patches: A1: The conductive ink is dispensed over the TPU sheet and let to dry. A2,3: The shape of the patch is cut using a desktop CO₂ laser. B1: A TPU sheet is laminated on top of the medical adhesive. B2,3: The patch shape and electrode holes are cut using a CO₂ laser. B4: The pre-etched flexible PCB is aligned with the previously cut substrate layers. C1: The two layers (A3 and B4) are aligned with the printed polymeric conductor facing the middle of the stack. C2: All layers are heat pressed together using a textile heat press at 150° C. for 30 s. C3: The rigid AFE PCB is aligned with the flexible PCB in the patch, and both are soldered together. The detail on the right shows a picture of the connection between the rigid AFE, the flexible PCB and the printed conductor. C4: For application in the user epidermis, the back paper of the adhesive is removed, and light pressure is applied between the patch and the user's skin to ensure strong adhesion between both.

FIG. 15: Schematic representation of a high level block diagram of the overall electrophysiology recording system.

FIG. 16: Shows the 3 electrode patches used for the impedance measurements.

FIG. 17: Cross-section of an embodiment of the skin electrode patch wherein A: Optical microscopy image of a cross-section of the fabricated patch showing that after the heat pressing process the electrode is aligned with the bottom adhesive layer enabling full contact with the skin. B: schematic of the photograph in A showing the relative position of each material layer. C: Close up optical microscopy image of the relative position between the two TPU layers and adhesive layer.

### DETAILED DESCRIPTION

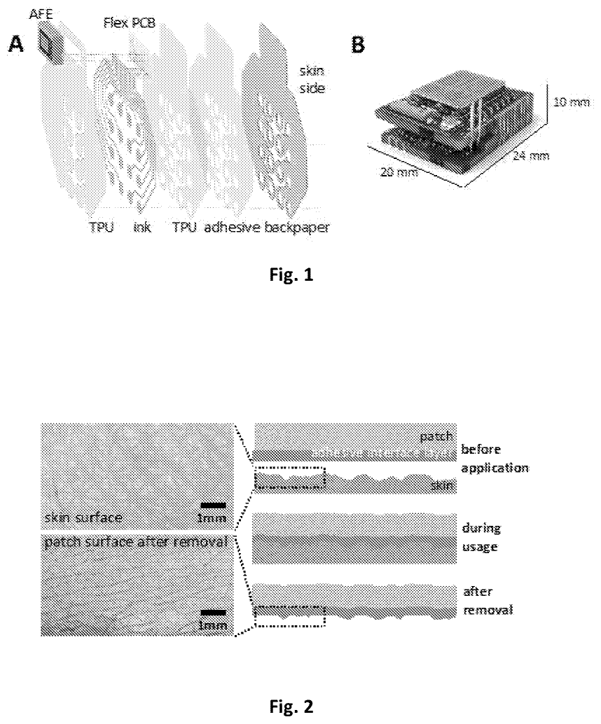

The present disclosure relates to a novel architecture of materials and methods for implementation of thin-film multielectrode adhesive patches for long-term and reliable monitoring of electrophysiological signals and digital biomarkers (FIG. 1A). It is shown that by using a bi-phasic Ag-EGaIn composite previously developed [28] and a multi-layer thin film (<210 μm) implementation, one can, thanks to a digital fabrication process, rapidly develop patient-specific multi-electrode biostickers, preferably the skin electrode patches, that seamlessly conform to the natural roughness and contours of the human skin and can be used for a range of biopotential recording applications. This includes single-lead ECG, which is also used to determining the respiration rate of the subject, multi-lead ECG, EOG, EEG, and EMG, during several days, while withstanding everyday activities such as jogging or bathing.

This is a fully standalone system, with the e-patch (FIG. 1A) is connected to a small-sized analog front-end (FIG. 1B) that also rests on the skin surface, allowing for true wireless biopotential monitoring of up to 16 electrodes.

Comprehensive study with 10 subjects shows that these electrodes provide a signal quality better than Ag/AgCl electrodes or than the same composite without liquid metal. Although these electrodes are solid-like and non-smearing [35], the inclusion of EGaIn droplets into the composite contributes to lower electrode-skin impedance, making this material an excellent choice for wearable epidermal electrodes as it combines the advantages of wet electrodes in terms of signal quality and skin-interfacing, and of dry electrodes (printability, low thickness and easy implementation).

Following, it will be disclosed the fabrication process.

FIG. 1A presents the layered structure of the multielectrode biopotential recording system. Referring to the figure, the system is composed of a soft patch attached to a rigid acquisition board.

The active layer of the biosticker consists of the printed conductive lines and skin interfacing electrodes made of Ag—In—Ga-SIS polymer [28]. The circuit is readily printed through direct ink writing and can be taylor-made for each user. The ink and electrodes can be printed with a resolution of <300 μm and thickness below 50 μm, thus allowing for implementation of high resolution multi-electrode bioelectronics. The active conductive layer is aligned with a thin, flexible interfacing printed circuit board and encapsulated between two layers of thermoplastic urethane (TPU; 50 μm thickness each) of the desired shape. All layers are fused together seamlessly through a heat pressing process similar to that which is used in t-shirt stamping. A pre-cut medical-gradeskin-compatible acrylic adhesive (60 μm thickness) with a backing paper liner is laminated to serve as the skin-adhesion layer prior to fusion. In the TPU and adhesive layers, holes were pre-patterned in the electrode locations to allow direct electrical contact between the ink and the skin. The rigid acquisition board, consists of the analog front end (AFE), processor and wireless communication module. The interface between the electronics and the patch is established through solder joints enabling a reliable mechanical and electrical connection between both.

A conformal and robust bond with the human skin is achieved by removing the adhesive's release liner and applying light pressure to the e-skin patch. Depending on the shape and placement of the e-skin in the human body, various distinct signals can be recorded, such as heart activity, brain activity, eye movement, respiration, or muscle activity in different locations. Detailed material listing and fabrication steps for both the soft e-skin patch and the rigid acquisition PCB are presented in the Methods section.

The comfort and usability of the developed system come not only from its lightweight (the weight of the AFE board is 5.99 g, while the weight of the patch is less than 8 g, depending on the application) but also from its reduced dimensions. The final patch has a maximum estimated thickness of ˜210 μm, corresponding to the laminated stack of two TPU layers, Ag—In—Ga-SIS ink, and medical-grade adhesive, while the rigid biopotential recording board measures only 20×24×10 mm, rendering it ideal for everyday use without impacting the user's movements.

Following it will be disclosed the characterization of the skin electrode patch.

It will be discussed now skin conformability and comfort.

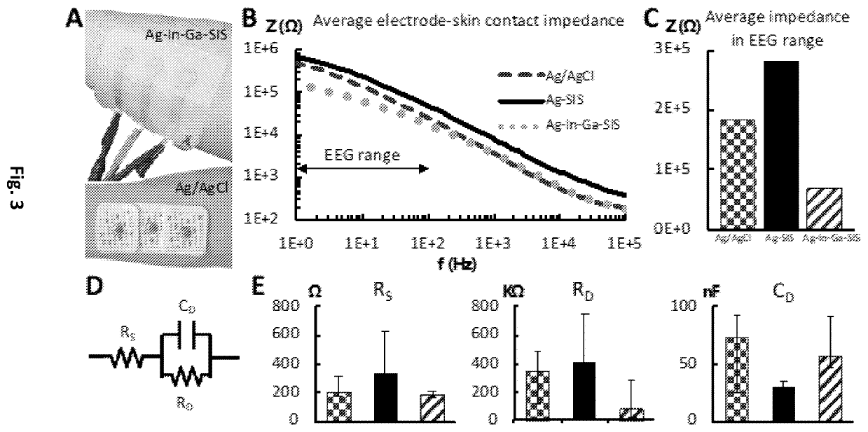

In an embodiment, to evaluate the conformability of the developed biostickers, one patch was adhered to the user's skin, and removed after one hour to be analyzed. As seen in FIG. 2A, the sub-millimeter wrinkles and creases of the skin surface were perfectly transferred to and replicated in the contact surface of the patch, showing the tight conformability of the ultrathin e-skin.

FIG. 2B shows a cross-section of the skin-electrode interface. The electrode perfectly adapts to the skin surface, creating a conformal contact area for reliable biopotential measurement.

FIG. 2C further shows the conformability of the developed e-skin patch, which can conform to the complex corrugated surface of an orange peel.

It was noted that the dynamic behaviour of the human skin is not affected by the e-skin patch, which keeps its conformal contact with the epidermis even under extreme deformation. The patch accompanies the skin movement without signs of delamination, as seen in FIG. 2D. Lastly, removal of the patch is similar to the removal of a traditional wound dressing, and while the adhesive leaves behind some redness due to skin-pulling, this fades away after no more than 30 minutes.

It will be discussed now the quality of electrode-skin interface.

In order to evaluate the suitability of the biostickers, the skin electrode patch, for acquisition of clinical grade biosignals, it was performed a human subject analysis to compare the quality of the skin-electrode interface when using the proposed ink electrodes, electrodes based on a similar polymer without EGaIn liquid metal, and clinical grade Ag/AgCl electrodes (FIG. 3A). The study involved 10 healthy adult subjects with no prior history of skin conditions (eczema, psoriasis, etc.) and no signs of erythema in the forearm region at the time of the study. The study consisted in capturing the Bode plots for the electrode-skin interface impedances (FIG. 9) measured in the right inner forearm for each subject in the 1-1ξ10⁵ Hz range for each electrode type. The ventral forearm was chosen for its accessibility and for being a region with low capillary density, following similar studies in the literature [40]. By observing the impedance data for the different subjects, we notice that for most subjects (S1 to S9), the measured impedances for all electrodes are between 102 and 106 Ω, and the bode plots show a smooth curve, while for subject 10, the measured impedances are between 10³ and 10⁸ Ω and the curves (mainly the ones regarding Ag-SIS and Ag—In—Ga-SIS electrodes) show spikes associated with noisy measurements.

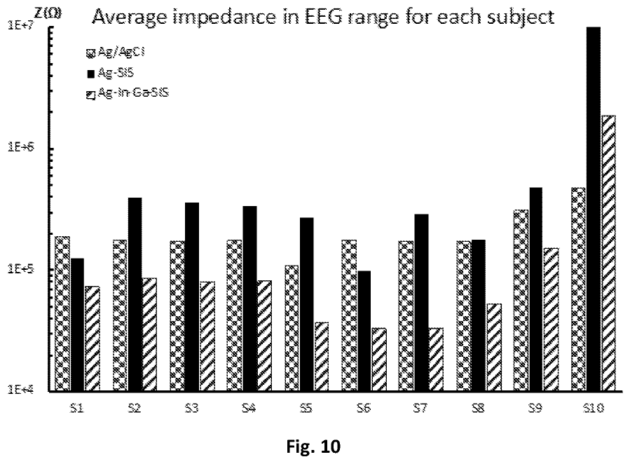

In the EEG range (1-100 Hz), shown in FIG. 10, the Ag—In—Ga-SIS electrodes show a lower impedance than the Ag/AgCl counterpart for subjects 1-9. In contrast, for subject 10, this trend is inverted, with Ag/AgCl showing the lowest impedance of the three compared materials. This outlier behaviour observed in subject 10 is linked with the fact that this subject presented a higher-than-average hair density in the tested site: the adhesive and gel-like properties of the Ag/AgCl electrodes allowed this material to flow between the hairs and maintain a decent contact with the subject's skin. In contrast, the Ag-SIS and Ag—In—Ga-SIS electrodes (non-adhesive per se) could not interface well with the underlying skin due to the high-density hair barrier leading to noisier and higher impedance measurements. Further analysis will focus solely on subjects 1 to 9, excluding the outlier, subject 10, concluding that the proposed electrodes are unsuitable for skin areas with extreme hair density. Individual measurements for each subject in the EEG range are plotted in FIG. 10. FIG. 3B shows the average electrode skin impedance plot for subjects 1-9, where we can observe that Ag—In—Ga-SIS electrodes show a lower impedance at lower frequencies, a trend observed in most of the tested subjects. The only case where this trend is not evident is with subject 9, in which the Ag/AgCl electrodes show a slightly lower impedance than the ink counterparts. In FIG. 3 C, the average electrode skin impedance in the EEG range (1-100 Hz), averaged for the first nine subjects, is shown, and the previous trend is confirmed: while Ag/AgCl show an average impedance of 1.84×10⁵±1.09×10⁵ Ω, the Ag—In—Ga-SIS electrodes show an impedance of only 6.99×10⁴±7.52×10⁴ Ω in the same range. The Ag-SIS polymer shows the highest impedance overall (2.81×10⁵±3.15×10⁵ Ω in the EEG range).

These results clearly show the advantage of inclusion of EGaIn liquid metal in the conductive composite. While this biphasic ink is printable and non-smearing, similar to other dry electrodes made with particle-filled composites, it has a considerably lower electrode-skin impedance, when compared to the same composite without EGaIn Liquid metal. Surprisingly, these electrodes showed also lower impedance than medical grade Ag/AgCl electrodes. This can be associated with several factors including the compliance of the electrode itself, and the thin-film structure of the biostickers—skin electrode patch—that adapts well to the wrinkles of the skin.

FIG. 3D depicts the equivalent electrical circuit for the electrode-skin interface proposed in [41], where Rs is linked to the resistance of the electrode material and the electrical path between electrodes through the living tissues, Rd corresponds to the interface resistance between the electrode and the skin, and Cd is the interface capacitance due to moving charges in the electrode-skin interface. The equivalent impedance for the presented circuit is given by Equation 1:

$$Ze = Rs + \frac{Rd}{1 + j 2 π fCd Rd} \qquad ( 1 )$$

From the literature [40], low Rd and Rs values and high Cd values are desirable to achieve the lowest possible impedance.

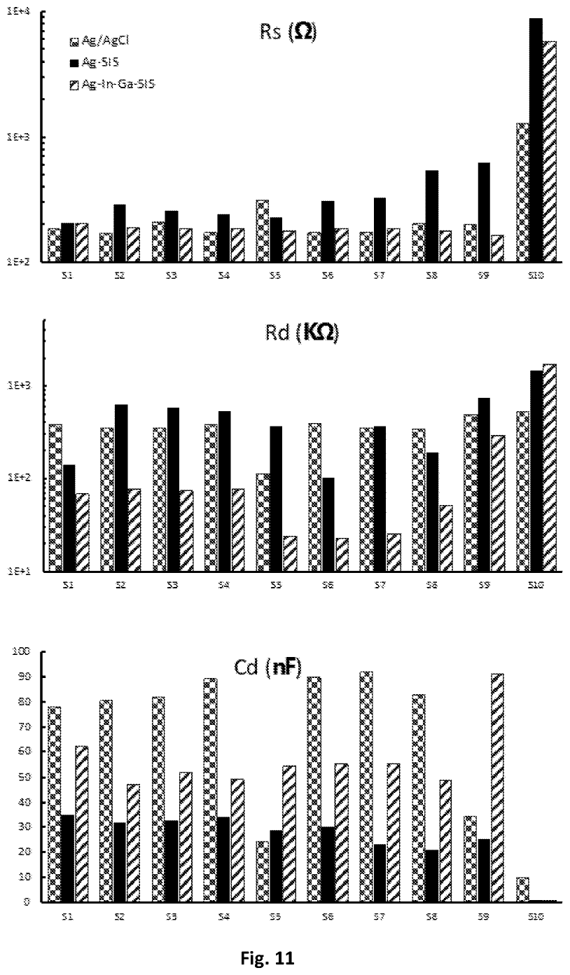

Using Equation 1, the circuit components (Rs, Cd, Rd) can be estimated from the Bode plot curves, as shown in FIG. 11 for each tested subject. FIG. 3E shows the average value of each component in each electrode type for the tested subjects (excluding the outlier subject 10). Regarding Rs, Ag—In—Ga-SIS electrodes show the lowest resistance (183.8±21.9 Ω), followed by Ag/AgCl (199.1±112.5 Ω) and finally Ag-SIS (334.6±286.9 Ω).

In terms of Rd, the trend is similar to Rs, with Ag—In—Ga-SIS electrodes showing the lowest value (78.8±212.6 K Ω), followed by Ag/AgCl (350.3±237.9 K Ω).and Ag-SIS (407.6±305.9 KΩ). In terms of Cd, Ag/AgCl electrodes present the best values (72.5±48.1 nF), followed by Ag—In—Ga-SIS (57.2±34.1 nF) and, lastly, Ag-SIS (29.1±8.2 nF).

The low resistances in the Ag—In—Ga-SIS electrodes can be explained by the high electrical conductivity of the composite itself (7.02×105 S m⁻¹³²) as well as the conformable interfacial contact of the patches with the skin. Compared to Ag-SIS, the biphasic composite (with Ga—In alloy) shows a much lower interface resistance which we attribute to the increase in conformability due to the presence of the Ga-base alloy, a liquid metal with high wettability [42].

In terms of the contact capacitance, the gel present in Ag/AgCl electrodes acts as a suitable dielectric in the electrode-skin bilayer and its intrinsic adhesiveness increases the contact area with the skin, resulting in the highest Cd value. The Ag—In—Ga-SIS, while not having a dielectric layer, still exhibits a capacitance that, although lower, is within the same order of magnitude as that of the Ag/AgCl. The comparable capacitance is attributed to the conformability and compliance of the material, which allows for increased contact area (proportional to the capacitance value as explained in [40]). The lower capacitance of Ag-SIS is attributed to the absence of Ga—In, since the liquid metal may improve the surface contact area between electrodes and the skin by filling microscopic interfacial gaps that can arise from the skin creases and roughness.

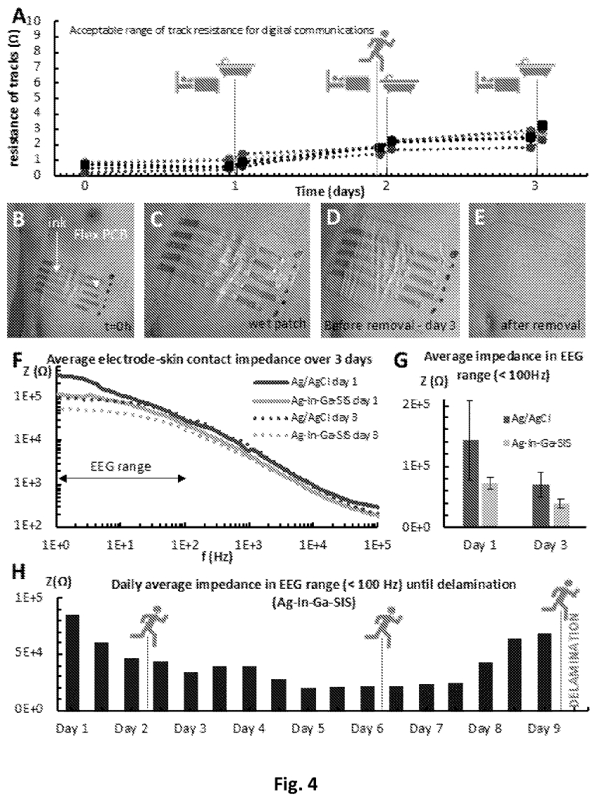

To understand the aging of Ag—In—Ga-SIS polymer, an adhesive patch with five printed tracks (80 mm length and 5 mm width) was fabricated and worn by a subject for three days. During these days, the subject did normal daily activities including sleeping, running and daily showering. At the end of day 3, the average line's resistance was 2.8 (±0.5) Ω, compared to 0.62 (±0.42) Ω at the beginning of the test, as can be observed in FIG. 4A. This value is still an acceptable value for digital circuits. As shown in the calculations presented in SI (equations S1 and S2), our circuits are functional even for a resistance of tracks of ˜10 Ω, For practical applications, such a slight increase in track resistance (which are much lower than the resistance observed in the electrode-skin interface) wouldn't affect the functionality or signal quality in the system.

FIG. 4 B shows the fabricated patch laminated in the user's abdomen.

The highest slope in the resistance curves is observed after bathing. This can be explained due to the TPU substrate of the patch swelling with humidity and bringing moisture in contact with the encapsulated ink. The wet patch is shown in FIG. 4C.

Lastly, no delamination was observed during the three days (FIG. 4D). When removing the patch, slight skin redness due to skin pulling is observable (FIG. 4E), which faded away in less than 30 minutes. Otherwise, no skin irritation or inflammation from adverse reaction to the biostickers materials was observed.

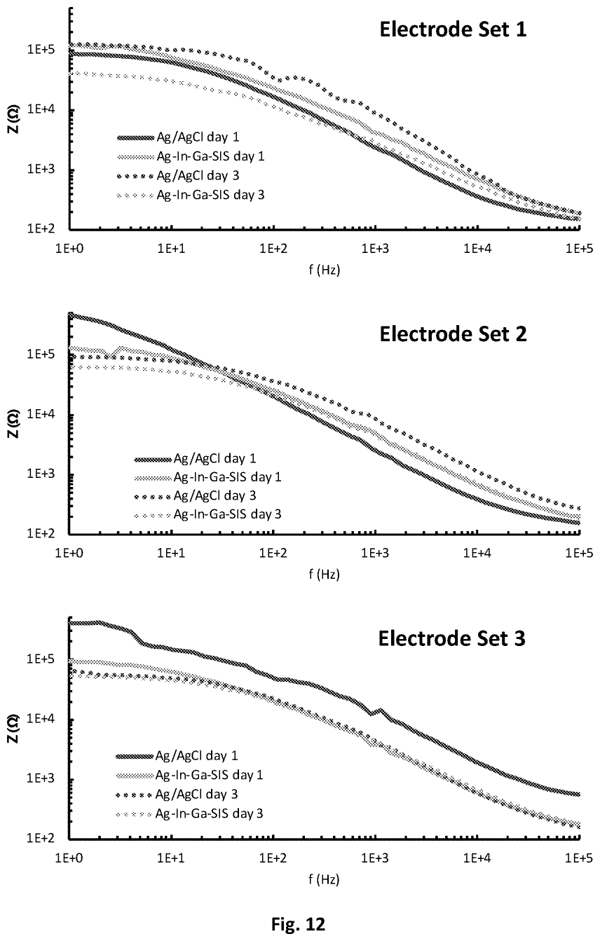

To evaluate the long-term electrical stability of the electrode skin interface, a user wore two distinct types of electrode sets (Ag—In—GA-SIS and Ag/AgCl) for three days. The electrode-skin impedance was measured for each electrode set after being initially placed and after being worn for 72 hours. A similar routine as before, including sleeping, running, and showering, was performed, and the test was repeated thrice on distinct days, with electrodes fabricated from separate batches of conductive composite. In FIG. 4F, the bode plot corresponding to the average electrode-skin impedance on the three trials is shown. The results for each trial are shown in FIG. 12. As shown in the large-scale trial with volunteers, right after application, the Ag—In—Ga-SIS electrodes show on average a lower interface impedance than the Ag/AgCl counterpart, which is more noticeable at lower frequencies. While the average impedance for the EEG range (FIG. 4F) of the Ag/AgCl electrodes on day 1 is 1.43×105 Ω, it is only 7.24×104 Ω for the polymeric electrodes. Contrary to what would be expected, none of the electrodes showed signs of degradation after three days, and the impedance of the electrode-skin interface decreases further. This observation was attributed to the buildup of sweat and moisture that gets trapped on the interface. This accumulation of electrolytes between the electrode and the skin further facilitates the movement of charges, thus decreasing impedance in the first couple of days. It is also relevant to note that the Ag/AgCl electrodes show a higher standard deviation of the impedance on the EEG range than the printed electrodes, which can be associated with the repeatability and stability of the Ag—In—Ga-SIS polymer and printing methodology across batches. Once again, no skin irritation or inflammation was observed in the sites where the Ag—In—Ga-SIS electrodes contacted the skin after three days of being worn.

Regarding possible applications, it will be shown now possible applications

The presented architecture of materials and fabrication techniques allow to rapidly fabricate biostickers for various applications, such as EMG, EEG, EOG or ECG. The possibility of digital printing permits to adjust the number and the geometry of the electrodes based on the applications and the user size. The external electronics remain the same for the various applications, while the biosticker can easily be changed and discarded.

Here, it is shown the acquisition of multiple digital biomarkers: single lead ECG, multi-lead ECG, Facial EMG, EOG, EEG and finally limb EMG.

It will be now disclosed Single-Lead Electrocardiography.

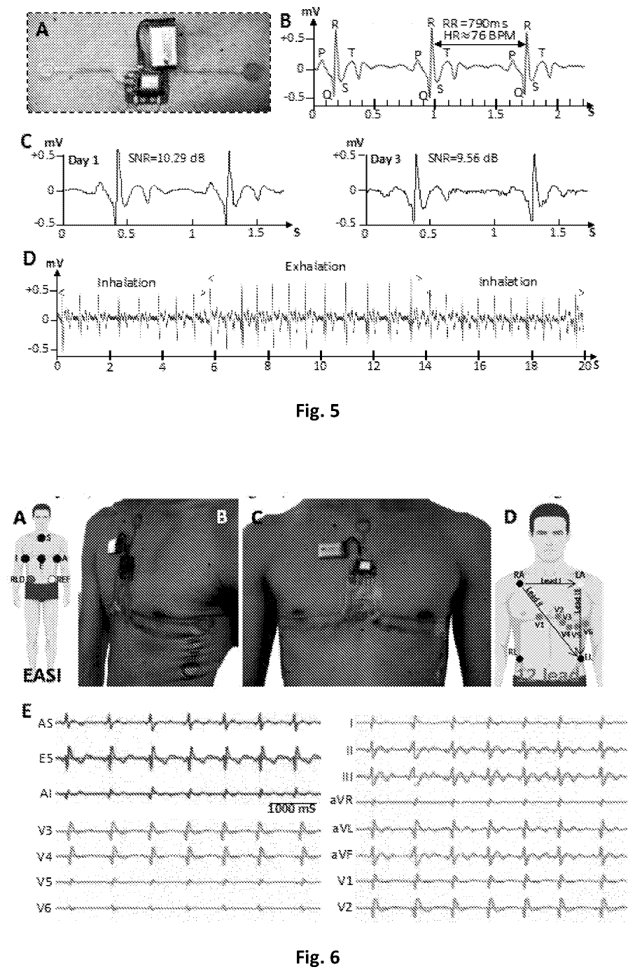

A one-lead ECG acquisition patch prototype was fabricated as the simplest use case scenario. As depicted in FIG. 5A, it is composed of two recording electrodes (FIG. 5B), one reference electrode, and a right leg drive electrode (RLD). FIG. 5C depicts a portion of the acquired ECG signal from a healthy subject, where the standard features of such a signal can be observed: the QRS complex, as well as P and T waves, are labeled in the figure. The RR interval (corresponding to the time between two consecutive R peaks) was calculated as being 790 ms, from which a heart rate of 76 bpm can be estimated. Furthermore, we can observe that the duration of the QRS complex is below 0.1 s and the time interval between Q and T waves falls below the limit of 0.4 s. From these characteristics, we were able to validate and infer the good quality of the recorded signal since all referred features and duration of waves fall within the limits stipulated by physicians for considering a normal ECG [43].

For this application, the subject wore the ECG patch for three days while keeping their usual daily routine. The signals recorded from day 1, after the patch was first adhered to the skin, and from after 72 hours are shown in FIG. 5D. Although the Signal to Noise Ratio (SNR) decreases slightly throughout the test duration (from −42.8950 dB to −51.1883 dB), all the typical ECG features are still visible on the signal recorded from day three and the RR interval, and heart rate can still be accurately calculated, despite the appearance of noise artifacts. Although the electrode-skin interface impedance decreases over the three days, the ink resistance was observed to increase and can explain the slight decrease in SNR.

It was also observed that the user's respiratory activity could be derived from the ECG recordings, as depicted in FIG. 5E. An increase in the amplitude of the QRS complex is related to exhalation. In contrast, when the user inhales, the amplitude of the QRS complex decreases. We hypothesize that this observation is due to the changes in impedance of the thoracic cavity during the respiration cycle, as well as to the stretching of the printed conductive lines acting a strain gauge, which lead to amplitude modulation of the ECG signal.

It will be now disclosed Multi-Lead Electrocardiography

As a slightly more complex use case, a multi-lead ECG patch was fabricated based on the EASI electrode configuration [44] (FIG. 6A-C). This system has the advantage of being able to derive all 12 lead measurements seen in a standard ten electrode ECG (FIG. 6D) by using only six electrodes and storing just three distinct signals instead of twelve. These characteristics make the EASI montage ideal for telemetry and wearable systems in which storage capacity or transmission bandwidth is limited. This also permits reducing the number of electrodes for easier deployment and long-term comfort of the user.

FIG. 6E depict in red the three leads acquired from the EASI montage (AS, ES, AI) and the derived 12 lead ECG in green. Features such as the QRS complex (normal sinus rhythm) can be observed in all signals, and as well the RR interval shows to be regular in all traces. These are characteristics of a normal adult 12-lead electrocardiogram, as stated in [45]. Apart from the regular RR interval (indicating the absence of arrhythmia [45]), the heart rate can also be calculated to be approximately 77 bpm, between the 60 and 100 lower and upper boundaries considered normal [45]. The equation and coefficients to perform the transformation between the EASI and the 12 lead ECGs are presented in the Methods section.

Regarding Electroencephalography, Electrooculography, and Facial EMG:

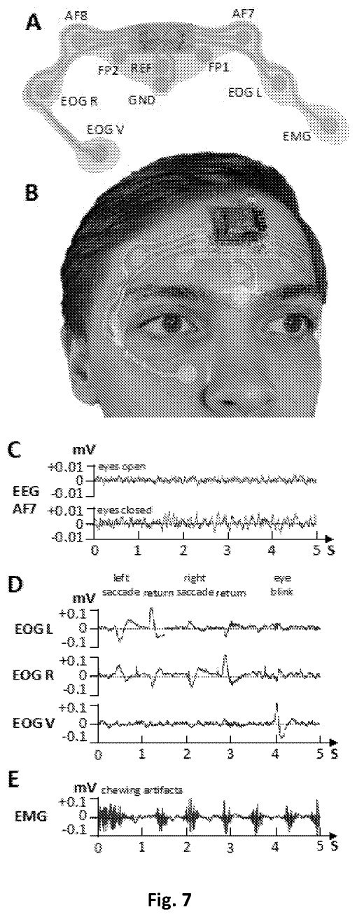

A patch containing ten electrodes was fabricated for recording brain activity, eye movement, and facial muscle activity. FIG. 7A depicts the placement and function of each electrode. FIG. 7B shows a user wearing the patch on their forehead and face. The simple and inexpensive digital fabrication process enables the fabrication of patient-specific patches with precise electrode positions and a custom layout that fits each user perfectly.

FIG. 7C shows an example of a recorded EEG signal in which the Berger effect [46] can be observed. This effect consists of suppression of the Alpha rhythm when the user opens their eyes, leading to a lower amplitude signal when compared to the brain activity while keeping their eyes closed. This observable difference in the two signals enables a clear separation between the two states. Although it is usually more noticeable in electrodes placed on the back of the scalp, we were still able to observe this with a signal acquired from the user's forehead.

FIG. 7D depicts EOG signals that were acquired. Observe that the left and right electrodes show opposite polarities when lateral eye movement is detected. On the other hand, eye blinks, predominantly vertical movements, are detected with higher amplitude by the EOG electrode placed below the user's eye. These are characterized by their high amplitude and low frequency.

Finally, sEMG artifacts derived from masticatory movement (FIG. 7E) are picked up by the electrode placed near the masseter muscle. These are characterized by being both high amplitude and high frequency. Overall, possible applications of this patch include fast deployable EEG exams for emergency or ambulatory settings, sleep monitoring and staging or detection, classification of facial expressions, and human/brain-machine interfaces.

Regarding Electromyography and Hand Pose Signature:

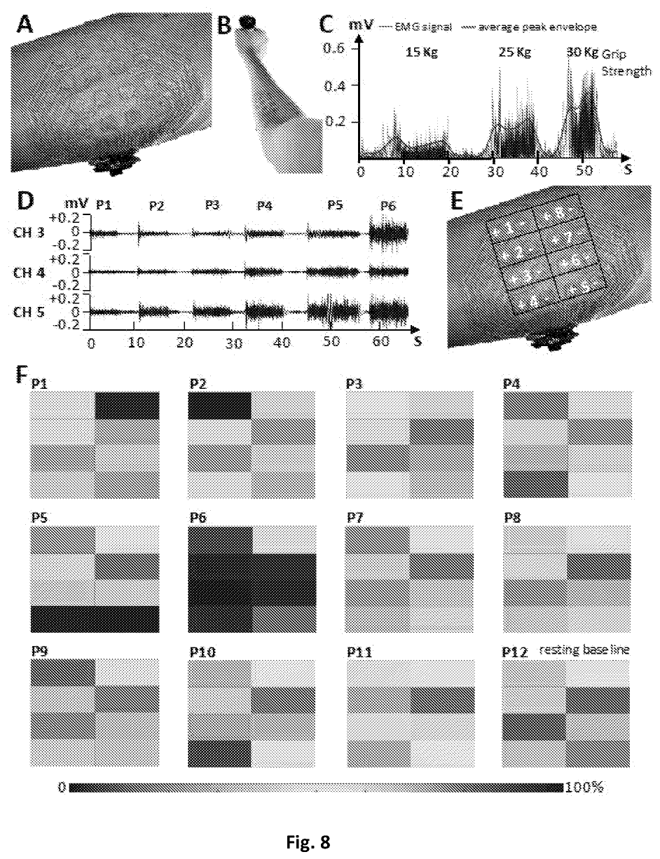

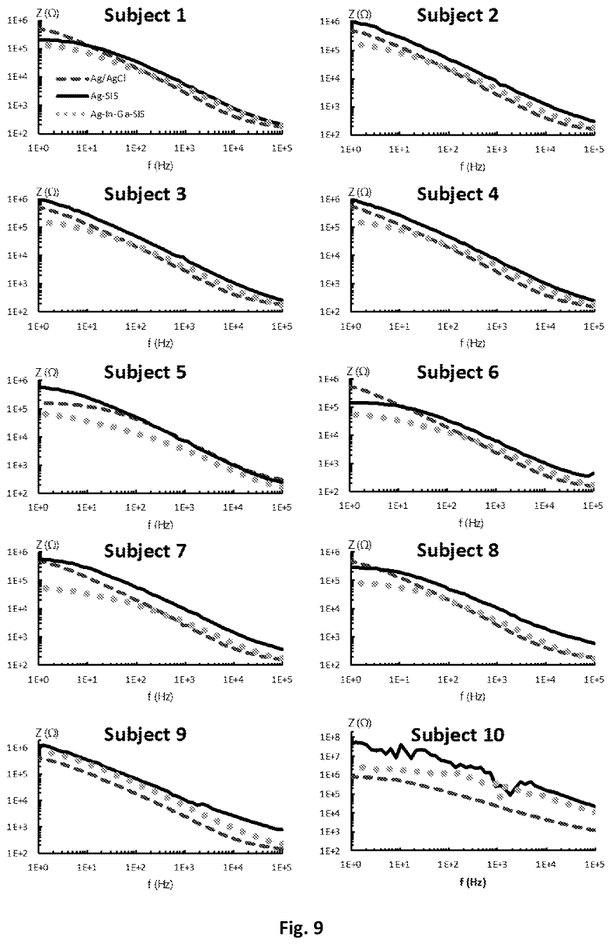

FIG. 8A shows a EMG patch with 17 electrodes. The patch is composed of 16 recording electrodes connected to 8 differential acquisition channels and placed on the proximal portion of the anterior right forearm of the user and a ground electrode that lays in the user's elbow. As a sanity check, a handgrip dynamometer (FIG. 8B) was used to measure the relation between EMG intensity and gripping strength. The results, shown in FIG. 8C, show that, as expected, the amplitude of the acquired and rectified EMG signal (in one of the channels) increases as the grip strength increases.

Taking advantage of the high channel count, we evaluated the feasibility of using the developed patch to detect hand gestures. In FIG. 8D, we can observe that for different hand poses (P1 to P6), the acquired EMG signals in each channel show distinct amplitudes (only three of the eight channels are shown). For simplicity, we chose to only use the average signal amplitude in each channel as the features to differentiate between hand-poses (in 1-second windows without overlapping). This simple feature vector has the advantage of being easy to extract and compute while generating small-sizeddatasets, thus being ideal for slow microcontroller-based systems where either power, processing, or memory are limited.

As shown in FIG. 8E, for each of the eight channels, the extracted average signal amplitude was scaled to a percentage where 100% corresponds to the maximum average amplitude detected in that channel.

FIG. 8F, which shows 12 distinct hand gestures and the respective amplitude footprint from the EMG signals in the eight channels. Overall, clear differences can be observed between most gesture pairs. The most similar cases can be observed to be the distinction between poses 4 and 10 and between poses 7 and 8, corresponding to cases where the gestures are similar, i.e. only showing differences in the position of one finger (fingers not mentioned being closed):
P1—index finger extended;
P2—index and middle fingers extended;
P3—index, middle and thumb fingers extended;
P4—index, middle, ring and little fingers extended;
P5—index, middle, ring, little and thumb fingers extended;
P6—closed fist;
P7—index, middle and ring fingers extended;
P8—index, middle and little fingers extended;
P9—index, ring and little fingers extended;
P10—middle, ring and little fingers extended;
P11—thumb finger extended;
P12—index, middle, ring, little and thumb fingers in open rest position.

FIG. 13A shows the dissimilarity coefficient between gesture pairs (computed using the Spearman correlation-based dissimilarity coefficient, i.e., the spearman distance between feature vectors of each pose observation, averaged across three one-second windows). While high dissimilarity values between pairs of gestures indicate an easy distinction between those poses (as is the case of the pair 6 and 12), dissimilarity coefficients as low as 0.1 [46] or 0.2 [48] have been previously shown good results in automatic EMG hand gesture classifiers. Overall, 85% of the gesture pairs show a dissimilarity coefficient above 0.2, while the average dissimilarity is 0.342. The average correlation coefficient for the same gesture across three repetitions (FIG. 13B) is above 0.75 for all 12 poses, meaning that the system shows good repeatability.

A similar work using an equivalent EMG electrode montage [48] has been able to distinguish only between 6 hand gestures with a classification accuracy of ˜95% on 12 participants. The present disclosure improves these results not only in terms of correlation coefficients, but also due to the use of a considerably simpler (in terms of features extracted from the EMG signal) and more wearable system. It is conjectured that the increased conformability and skin compliance of our system, as well as the low electrode-skin impedance, leads to a better acquired EMG signal and thus a more robust distinction between a larger set of gestures. As such, a good gesture classification accuracy using machine learning algorithms would be expected for our 12-poses dictionary.

It is disclosed a disposable ultrathin multi-layer adhesive patch with digitally printed skin-interfacing electrodes and interconnects based on an Ag—In—Ga filled elastomer, which conforms tightly to the natural wrinkles and creases of the human skin even during normal deformation. With the digital printing, the presented technique can be easily scaled up for rapid fabrication of taylor-made biostickers patches for different electrophysiological monitoring applications. It is shown that the developed patches can be worn for several days without interfering with the quality of signal acquisition. It is not observed any significant deterioration of the biphasic composite after 3 days of continuous wearing, and the electrode skin impedance even decreased after three days. During this test, the user performed his normal daily routine, including showering, running, and other everyday activities.

In an embodiment, the printed patch attaches directly to a reusable small-sized analog front end which communicates the acquired data in real-time via WiFi. Depending on the patch shape and its positioning in the body, different applications were shown: First, a simple 1-lead ECG monitoring setup was presented. The relevant features of the ECG wave could be correctly observed after using the patch for 3 days, while doing normal daily routines such as running, and taking bath. It was also shown that this system could also monitor the user's respiration pattern, which can be inferred from the ECG amplitude, making this patch a good candidate for sleep apnea monitoring and detection.

This was then extended to a 12 lead ECG, though he EASI electrode montage. Once again, the ECG wave features are observable in the acquired data. Possible use case scenarios include long-term Holter-like heart monitoring with the advantage of the patient being able to shower. Using a patch that covers the user's forehead and face, we could also record brain activity, eye motion, and muscle activity from chewing. Finally, a patch for recording limb electromyography was fabricated, and the identification of 12 distinct hand gestures was shown. We showed that the fingerprint of the 12 poses are distinct enough from each other to enable good performance of automatic classifiers, and presented improved result compared to the state of the art. For the same gesture, the acquired signals show good repeatability (average similarity coefficient of each pose across three repetitions is above 0.7 for all 12 poses for the same subject). These correlation metrics can be further improved by extracting more complex features from the recorded EMG data (for instance, features in the frequency domain) at the cost of increased computation complexity and memory requirements.

A human subject trial was performed on ten volunteers to assess the quality of the conductive polymer-based skin-interfacing electrodes. It was observed that the proposed polymeric electrodes show an average impedance in the 1-100 Hz range lower than the Ag/AgCl counterpart, which is attributed to the high conductivity of the composite itself, as well as to the excellent skin-conformability of the fabricated patches, given that the electrodes are not placed in a region with high capillary density. The Ga—In alloy used in the conductive composite was also shown to have a decisive role in improving the quality of the interface by decreasing the electrode-skin impedance when compared with a simple particle-filled Ag-SIS polymer. Furthermore, none of the tested subjects reported any adverse skin reaction or inflammation from contact with the Ag—In—Ga-SIS electrodes. In one case, when the hair density on the test zone was high, the Ag/AgCl electrode performed better than our printed electrodes.

It is also shown that the bioelectronic patches can be worn for several days without interfering with the quality of signal acquisition and without affecting the user's routine. No deterioration of the conductive polymer was observed and the electrode skin impedance was even found to decrease after three days. In particular, the patch was able to withstand daily showering, running, and other everyday activities, suggesting that these activities are feasible when wearing the adhesive electrode patch and have no noticeable impact on its integrity. Furthermore, none of the tested subjects reported any adverse skin reaction or inflammation from contact with the Ag—In—Ga-SIS electrodes.

Overall, the proposed biostickers or patches disclosed, combines excellent user comfort, durability, excellent signal quality, and possibility of direct digital printing for scalable fabrication of taylor-made patches. This advancement can pave an important step, toward untethering the patients from bulky devices, and toward domiciliary hospitalization, while enabling clinical grade remote patient monitoring.

The following paragraphs discloses the methods to obtain the patch.

In an embodiment, to obtain the conductive ink preparation the following steps were performed.

It started by synthesizing the conductive Ag—In—Ga-SIS ink as presented in [28]. First SIS (Styrene-Isoprene Copolymer—Aldrich Chemistry) is diluted in Toluene (1:3 wt. %). Silver flakes (Ag071 Technic inc.) are mixed into the SIS solution (2:1 wt. %) using a planetary mixer (Thinky ARE-250) for 3 minutes at 2000 rpm. Previously prepared EGaIn (75.5% Ga, 24.5% In) is mixed into the Ag-SIS solution in 2:1 EGaIn:Ag wt. % ratio, and the solution is mixed in the planetary mixer for 3 minutes at 2000 rpm. The resulting Ag—In—Ga-SIS solution—now with a shiny silvery color—is loaded into a syringe barrel for further printing. For the electrode-skin-impedance tests a similar formulation of the ink but without the liquid metal was also synthesized for comparison purposes.

In an embodiment, the E-patch fabrication was made according with the following steps.

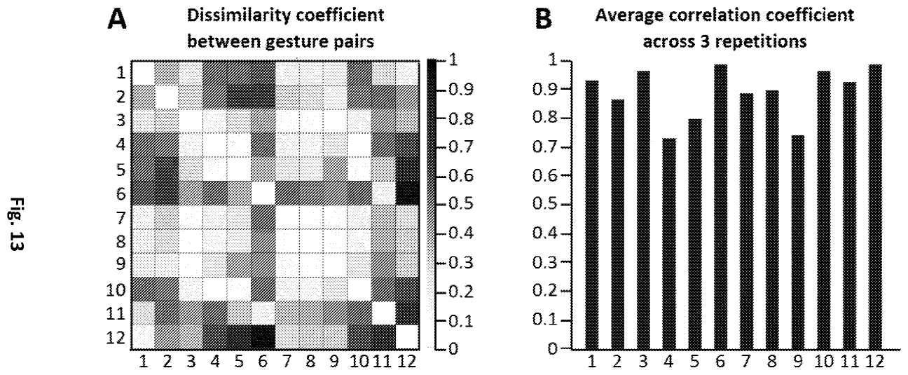

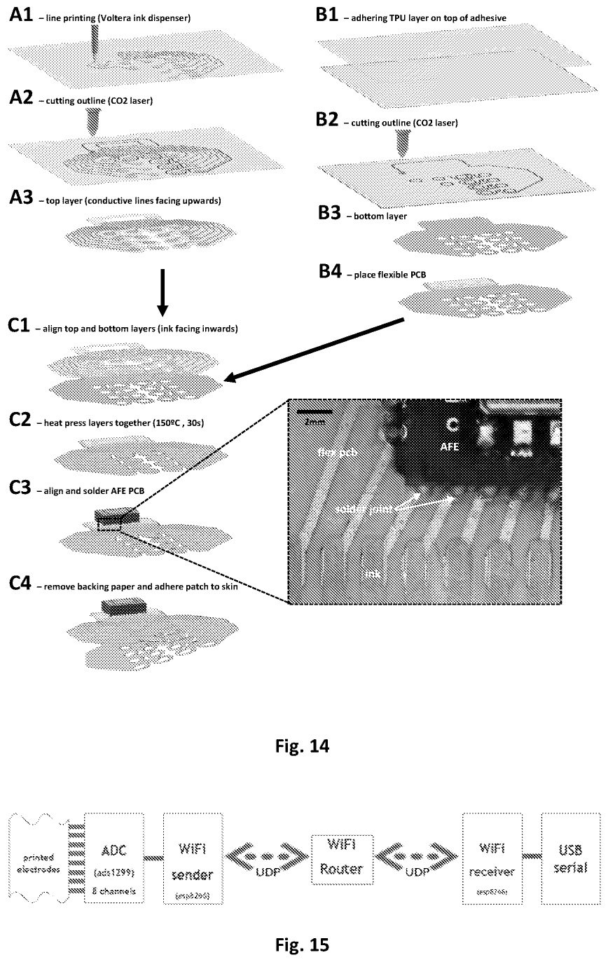

The Ag—In—Ga-SIS ink is dispensed over a TPU film (Bemis 3412 TPU hotmelt film) following the previously designed paths for the electrodes and interconnects, using a Voltera ink dispensing system (Voltera V-One PCB printer), and the film is let to dry at 60° C. for 10 minutes (FIG. 14A1). The outline of the patch is then cut using a CO₂ Laser (Universal Laser Systems VLS 3.50), as seen in FIG. 14A2, leaving the top part of the patch ready (FIG. 14A3). For the bottom part of the patch, a TPU film is adhered on top of a medical-grade skin-compatible adhesive with back paper (3M 1524A Medical Transfer Adhesive)—FIG. 14B1—and the outline is cut with the CO₂ laser—FIG. 14B2, B3. A previously etched interfacing flexible Kapton-copper PCB is then aligned and placed on top of the TPU-adhesive layer, as seen in FIG. 14B4. The two prepared parts (top and bottom layer) are aligned together with the printed lines facing inwards (FIG. 14C1) and heat pressed together (FIG. 14C2) for 30 seconds at 150° C. using a t-shirt printing press machine (Surpcos HPM-121505). The rigid acquisition PCB is aligned with the flexible PCB in the patch, and both are soldered together, as seen in FIG. 14C3. Finally, the backing paper of the adhesive can be removed (FIG. 14C4), and the system is ready to be laminated to the user's skin.

Regarding the biopotential acquisition, communication, and signal filtering:

The rigid circuit board that attaches to the patch measures 20×24×10 mm, and it weighs only 5.99 g. This PCB is composed of an acquisition layer and a communication layer. The signal acquisition and amplification rely on the ADS1299 module (ADS1299, Texas Instruments), an 8-channel differential amplifier with 24 bit ADC. In our setup, signals are sampled at 250 Hz and amplified 24×, enough for recording even the lowest amplitude surface biopotentials (EEG). The system was powered using a 3.7V LiPo Battery, and its current consumption during normal function is below 50 mA. The digitized signals are then passed on to the microcontroller (ESP8266 Expressif Systems), which sends them through the WiFi network (using a UDP protocol) to a receiver (based on the same microcontroller). The receiver dongle sends the incoming values from the WiFi network to the serial port for visualization, recording, and further processing, as seen in FIG. 15. The incoming signals from the serial port are saved and later filtered in MATLAB using a notch filter (60 Hz) and High/Low pass second-order Butterworth filters as needed for each monitored signal. For ECG, the relevant frequencies were assumed to be in the 5-55 Hz band. For EEG, EOG, and EMG, the relevant frequencies were considered to be in the 2-100 Hz band. The 12-lead ECG signals (v) can be derived from the acquired EASI potentials (v_{ES}, v_{AS}, v_{AI}) through the following equation:

$$v = a \cdot {v}_{ES} + b \cdot {v}_{AS} + c \cdot {v}_{AI} \qquad ( 2 )$$

The fixed coefficients (a, b, c) for the calculation of each lead are shown in Table 1.

TABLE 1
Transformation coefficients for approximation of
the 12-lead ECG through acquired EASI signals

| Lead (ν) | a | b | c |
|---|---|---|---|
| I | 0.026 | −0.174 | 0.701 |
| II | −0.002 | 1.098 | −0.763 |
| III | −0.028 | 1.272 | −1.464 |
| aVR | −0.012 | −0.462 | 0.031 |
| aVL | 0.027 | −0.723 | 1.082 |
| aVF | −0.015 | 1.185 | 1.114 |
| V1 | 0.641 | −0.391 | 0.080 |
| V2 | 1.229 | −1.050 | 1.021 |
| V3 | 0.947 | −0.539 | 0.987 |
| V4 | 0.525 | 0.004 | 0.841 |
| V5 | 0.179 | 0.278 | 0.630 |
| V6 | −0.043 | 0.431 | 0.213 |

Following it will be disclosed the electrode-skin impedance measurement.

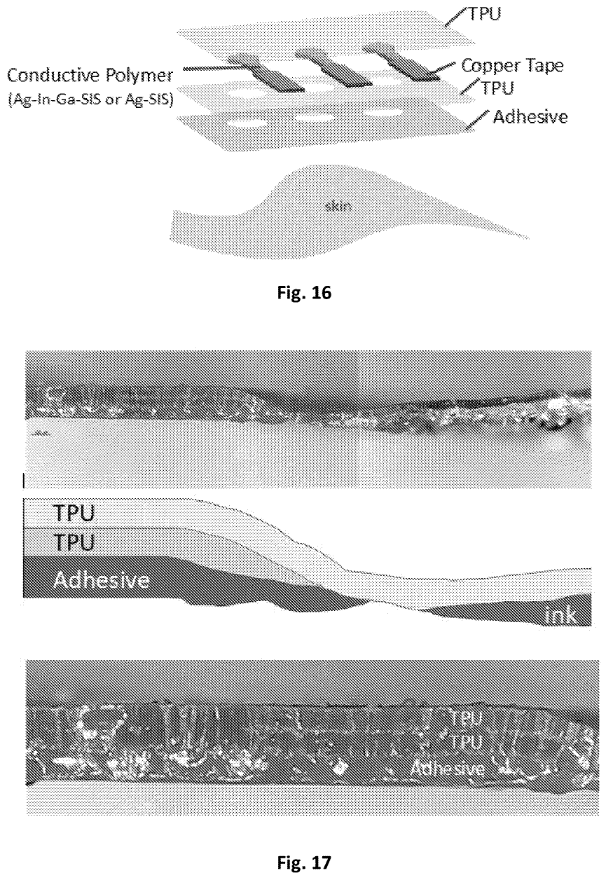

In an embodiment, patches with 3 Ag—In—Ga-SIS electrodes were fabricated (FIG. 16), as well as patches with Ag-SIS (without the liquid metal). The impedance on these, as well as Ag/AgCl electrodes (3M Red Dot) was measured, using a PalmSens4 impedance analyzer. 50 impedance points (approximately 9.8/decade) were measured between 10⁰ and 10⁵ Hz for each bode plot.

Before placing each set of electrodes on the right inner forearm of the volunteer, the skin was cleaned by wiping it with rubbing alcohol and letting it dry for 1 minute. The electrode set to be recorded was then placed and left to rest for 1 minute, and the impedance measurement was taken. After the measurement was complete, the electrodes were removed, and any residue of adhesive was wiped off with rubbing alcohol. The process was then repeated for other electrodes. The equivalent circuit components for the electrode-skin interface were approximated by fitting the obtained bode plots to the equivalent impedance expression through the Levenberg-Marquardt algorithm (damped least-squares), integrated into the PalmSens PSTrace software.

Ten volunteers participated in this study, which was approved by the Carnegie Mellon University Institutional Review Board (STUDY2022_00000015) in accordance with the US HHS regulations for the protection of human subjects in research at 45CFR 46. Informed consent was obtained from all the participants, and all experiments were performed in accordance with the applicable regulations.

Following it will be disclosed SNR calculations.

In an embodiment, after filtering the acquired ECG signals, signal-to-noise ratio calculations were performed in Matlab. The function used for the calculation, snr(s,n), is based on the formula from Equation 3:

$$20 {log}_{10} ( \frac{rssq ( s )}{rssq ( n )} ) \qquad ( 3 )$$

where rssq(s) corresponds to the root-sum-of-squares of the clean signal amplitude and rssq(n) corresponds to the root-sum-of-squares of the removed noise amplitude.

Following it will be disclosed acceptable range of resistance for printed conductive tracks:

For digital communications, considering a system that works in a 3.3 V voltage level and where the cutline for a high logic level is 2.475 V, the maximum acceptable voltage drop in any line would be 0.825 V (as is the case of the used microcontroller ESP8266). Suppose the same system can deliver a 12 mA current in any digital port (as is the case of ESP8266). In that case, the maximum acceptable resistance of a given digital circuit is given by Equation S1.

$${R}_{max} = \frac{Δ {V}_{max}}{{I}_{max}} = \frac{0.8 \; 25 V}{12 mA} ⇔ {R}_{max} = 68.75 Ω \qquad ( S1 )$$

Since the circuit must be composed of at least 2 lines (signal line and ground return path), the maximum admissible resistance of each track, independently of line length and line stretching, is given by Equation S2:

$${R}_{max / track )} = \frac{{R}_{max}}{2} = 34.3 \; 75 Ω \qquad ( S2 )$$

For the sake of signal integrity and to avoid data corruption in all circumstances (even when the lines are stretched), let us consider that the maximum admissible resistance of each of the tracks we tested is approximately ⅓ of the previous value, i.e., 10 Ω. As observed in FIG. 4A, the slight increase in resistance in the tested lines is well below the 10 Ω threshold, so the printed polymer could still be used reliably as a conductor in any digital circuit.

In the case of the disclosed system, the conductive ink is only used to conduct analog biopotential signals, which are not significantly affected by slight resistance changes in the Ohms range, which are much lower than the resistance observed in the electrode-skin interface.

Support for this research was provided by the Fundação para a Ciência e a Tecnologia (Portuguese Foundation for Science and Technology) through the Carnegie Mellon Portugal Program under Grant SFRH/BD/150691/2020. Funding also came from the CMU-Portugal project WoW (45913), which had the support of the European Regional Development Fund (ERDF) and the Portuguese State through Portugal2020 and COMPETE 2020, as well as the Nano-Bio Materials Consortium (NBMC), sponsored by the US Air Force Research Laboratory (AFRL) in partnership with SEMI.

The term “comprising” whenever used in this document is intended to indicate the presence of stated features, integers, steps, components, but not to preclude the presence or addition of one or more other features, integers, steps, components or groups thereof.

The disclosure should not be seen in any way restricted to the embodiments described and a person with ordinary skill in the art will foresee many possibilities to modifications thereof. The above described embodiments are combinable.

The following claims further set out particular embodiments of the disclosure.

### References

1. Iqbal, S. M. A., Mahgoub, I., Du, E., Leavitt, M. A. & Asghar, W. Advances in healthcare wearable devices. npj Flexible Electronics vol. 5 (2021).
2. Yokus, M. A. & Jur, J. S. Fabric-based wearable dry electrodes for body surface biopotential recording. IEEE Trans. Biomed. Eng. 63, 423-430 (2016).
3. Alberto, J. et al. Fully Untethered Battery-free Biomonitoring Electronic Tattoo with Wireless Energy Harvesting. Sci. Rep. (2020) doi:10.1038/s41598-020-62097-6.
4. Wang, H. et al. Robust tattoo electrode prepared by paper-assisted water transfer printing for wearable health monitoring. IEEE Sens. J. (2022) doi:10.1109/JSEN.2022.3141457.
5. Green Marques, D., Alhais Lopes, P., De Almeida, A. T., Majidi, C. & Tavakoli, M. Reliable interfaces for EGaIn multi-layer stretchable circuits and microelectronics. Lab Chip (2019) doi:10.1039/c8lc01093e.
6. Tavakoli, M., Benussi, C. & Lourenco, J. L. Single channel surface EMG control of advanced prosthetic hands: A simple, low cost and efficient approach. Expert Syst. Appl. (2017) doi:10.1016/j.eswa.2017.03.012.
7. Paul, G. M. et al. A Smart Textile Based Facial EMG and EOG Computer Interface. IEEE Sensors 14, 393-400 (2014).
8. Myllymaa, S. et al. New disposable forehead electrode set with excellent signal quality and imaging compatibility. J. Neurosci. Methods 215, 103-109 (2013).
9. Looney, D. et al. The in-the-ear recording concept: User-centered and wearable brain monitoring. IEEE Pulse 3, 32-42 (2012).
10. Carneiro, M. R., De Almeida, A. T. & Tavakoli, M. Wearable and comfortable e-textile headband for long-term acquisition of forehead EEG signals. IEEE Sens. J. 1-1 (2020) doi:10.1109/JSEN.2020.3009629.
11. Lepola, P. et al. Screen-printed EEG electrode set for emergency use. Sensors Actuators, A Phys. 213, 19-26 (2014).
12. Ameri, S. K. et al. Imperceptible electrooculography graphene sensor system for human-robot interface. npj 2D Mater. Appl. 2, 1-7 (2018).
13. Carneiro, M. R. & Tavakoli, M. Wearable Pressure Mapping through Piezoresistive C-PU Foam and Tailor-Made Stretchable e-Textile. IEEE Sens. J. 21, (2021).
14. Sang, Z., Ke, K. & Manas-Zloczower, I. Design Strategy for Porous Composites Aimed at Pressure Sensor Application. Small 15, 1-9 (2019).
15. Patron, D. et al. On the Use of Knitted Antennas and Inductively Coupled RFID Tags for Wearable Applications. IEEE Trans. Biomed. Circuits Syst. 10, 1047-1057 (2016).
16. Gao, W. et al. Flexible Electronics toward Wearable Sensing. Acc. Chem. Res. 52, 523-533 (2019).
17. Kabiri Ameri, S. et al. Graphene Electronic Tattoo Sensors. ACS Nano 11, 7634-7641 (2017).
18. Wang, G. et al. Stretchable Optical Sensing Patch System Integrated Heart Rate, Pulse Oxygen Saturation, and Sweat pH Detection. IEEE Trans. Biomed. Eng. 66, 1000-1005 (2019).
19. Santos, L. et al. Dynamic Gesture Recognition Using a Smart Glove in Hand-Assisted Laparoscopic Surgery. Technologies 6, 8 (2018).
20. Jiao, Y. et al. Wearable Graphene Sensors with Microfluidic Liquid Metal Wiring for Structural Health Monitoring and Human Body Motion Sensing. IEEE Sens. J. 16, (2016).
21. Silva, A. F. & Tavakoli, M. Domiciliary hospitalization through wearable biomonitoring patches: Recent advances, technical challenges, and the relation to covid-19. Sensors (Switzerland) (2020) doi:10.3390/s20236835.
22. Buhagiar, M. A. et al. Effect of inpatient rehabilitation vs a monitored home-based program on mobility in patients with total knee arthroplasty the HIHO randomized clinical trial. JAMA—J. Am. Med. Assoc. 317, (2017).
23. Levine, D. M. et al. Hospital-level care at home for acutely ill adults a randomized controlled trial. Ann. Intern. Med. 172, (2020).
24. Hong, S., Lee, S. & Kim, D. H. Materials and Design Strategies of Stretchable Electrodes for Electronic Skin and its Applications. Proceedings of the IEEE vol. 107 (2019).
25. Zhao, Y., Kim, A., Wan, G. & Tee, B. C. K. Design and applications of stretchable and self-healable conductors for soft electronics. Nano Convergence vol. 6 (2019).
26. Kim, D.-H. et al. Epidermal Electronics. www.sciencemag.org.
27. Tavakoli, M. et al. EGaIn-Assisted Room-Temperature Sintering of Silver Nanoparticles for Stretchable, Inkjet-Printed, Thin-Film Electronics. Adv. Mater. (2018)
28. Lopes, P. et al. Bi-Phasic Ag—In—Ga Embedded Elastomer inks for Digitally Printed, Ultra-Stretchable, Multi-Layer Electronics. ACS Appl. Mater. Interfaces 13, 14552-14561 (2021).
29. Wu, W. Stretchable electronics: functional materials, fabrication strategies and applications. Science and Technology of Advanced Materials vol. 20 187-224 (2019).
30. Kazem, N., Hellebrekers, T. & Majidi, C. Soft Multifunctional Composites and Emulsions with Liquid Metals. Advanced Materials vol. 29 (2017).
31. Liu, S., Shah, D. S. & Kramer-Bottiglio, R. Highly stretchable multilayer electronic circuits using biphasic gallium-indium. Nat. Mater. 20, (2021).
32. Rocha, R. P. et al. Fabrication and characterization of bending and pressure sensors for a soft prosthetic hand. J. Micromechanics Microengineering 28, (2018).
33. Wissman, J., Lu, T. & Majidi, C. Soft-matter electronics with stencil lithography. in Proceedings of IEEE Sensors (IEEE Computer Society, 2013).
34. Lu, T., Finkenauer, L., Wissman, J. & Majidi, C. Rapid prototyping for soft-matter electronics. Adv. Funct. Mater. 24, 3351-3356 (2014).
35. Reis Carneiro, M., Majidi, C. & Tavakoli, M. Dielectric Elastomer Actuators with Biphasic Ag-EGaIn Electrodes. (2021) doi:10.1002/adem.202100953.
36. Lopes, P. A. et al. Reversible polymer-gel transition for ultra-stretchable chip-integrated circuits through self-soldering and self-coating and self-healing. Nat. Commun. 12, (2021).
37. Carvalho, F. M. et al. Nondrying, sticky hydrogels for the next generation of high-resolution conformable bioelectronics. ACS Appl. Electron. Mater. 2, (2020).
38. Ohm, Y. et al. Publisher Correction: An electrically conductive silver-polyacrylamide-alginate hydrogel composite for soft electronics. Nat. Electron. 4, (2021).
39. Zhang, L. et al. Fully organic compliant dry electrodes self-adhesive to skin for long-term motion-robust epidermal biopotential monitoring. Nat. Commun. 11, (2020).
40. Lopes, P. A. et al. Soft Bioelectronic Stickers: Selection and Evaluation of Skin-Interfacing Electrodes. Adv. Healthc. Mater. (2019) doi:10.1002/adhm.201900234.
41. Albulbul, A. Evaluating major electrode types for idle biological signal measurements for modern medical technology. Bioengineering 3, (2016).
42. Doudrick, K. et al. Different shades of oxide: From nanoscale wetting mechanisms to contact printing of gallium-based liquid metals. Langmuir 30, (2014).
43. Yanowitz, F. G. Introduction to ECG Interpretation. (Intermountain Healthcare, 2018).
44. Ng, J. & Goldberg, J. J. The Physiological Measurement Handbook—Cardiology. (CRC Press, 2014). doi:10.1201/b17831-4.
45. Jenkins, D. & Gerred, S. Normal adult 12-lead ECG. ECG Library. https://ecglibrary.com/norm.php.
46. Kirschfeld, K. The physical basis of alpha waves in the electroencephalogram and the origin of the ‘berger effect’. Biological Cybernetics vol. 92 (2005).
47. Rabbi, M. F. et al. Non-negative matrix factorisation is the most appropriate method for extraction of muscle synergies in walking and running. Sci. Rep. 10, (2020).
48. Rosati, G. et al. Inkjet-printed fully customizable and low-cost electrodes matrix for gesture recognition. Sci. Rep. 11, (2021).

1. A method for obtaining a skin electrode patch, comprising the steps of:
applying conductive ink to a first polymeric layer;
cutting one or more cut-outs, on a second polymeric layer, for the conductive ink to contact with the skin;
arranging as consecutive layers:
the first polymeric layer;
the conductive ink;
a flexible printed circuit board (“PCB”); and
the second polymeric layer; and
heat-pressing the arranged layers,
wherein the conductive ink is configured to be exposed through said one or more cut-outs to the skin and to connect with the flexible PCB.

2. The method according to claim 1, further comprising arranging an adhesive layer on said second polymeric layer, said adhesive layer having one or more cut-outs for the conductive ink to contact with the skin.

3. The method according to claim 1, further comprising arranging said adhesive layer as a consecutive layer on the second polymeric layer before heat-pressing the arranged layers.

4. The method according to claim 1, wherein the heat-pressing is performed with a textile heat press.

5. The method according to claim 1, wherein the temperature of the heat-pressing is at least 150° C.

6. The method according to claim 1, further comprising arranging a rigid printed circuit board, on the flexible PCB.

7. The method according to claim 1, wherein the cutting is performed by a laser.

8. The method according to claim 1, wherein the first polymeric layer and the second polymeric layer are made of thermoplastic polyurethane.

9. The method according to claim 1, wherein the conductive ink is a Silver—Indium—Gallium—Styrene-Isoprene Copolymer (“Ag—In—Ga-SIS”) ink.

10. A skin electrode patch comprising consecutive heat-pressed layers of:
a first polymeric layer;
conductive ink;
a flexible printed circuit board (“PCB”); and
a second polymeric layer,
wherein the second polymeric layer has one or more cut-outs for the conductive ink to contact with the skin, and
wherein the conductive ink is configured to be exposed through said one or more cut-outs to the skin and to connect with the flexible PCB.

11. The skin electrode patch according to claim 10 further comprising an adhesive layer on said second polymeric layer, having one or more cut-outs for the conductive ink to contact with the skin.

12. The skin electrode patch according to claim 10, wherein the cut-outs of the first polymeric layer correspond to the cut-outs of the adhesive layer.

13. The skin electrode patch according to claim 10, wherein a rigid printed circuit board, is arranged on the flexible PCB.

14. The skin electrode patch according to claim 10, wherein the first polymeric layer and the second polymeric layer has a thickness of 50 μm each.

15. The skin electrode patch according to claim 11, wherein the adhesive layer has a thickness of 60 μm.

16. The skin electrode patch according to claim 10, wherein the conductive ink is a Silver—Indium—Gallium—Styrene-Isoprene Copolymer (Ag—In—Ga-SIS″) ink.

17. The skin electrode patch according to claim 10, wherein the first polymer layer and the second polymer layer are made of thermoplastic polyurethane.
